(19) United States
(12) Patent Application Publication
(10) Pub. No.: US 2012/0296140 A1
(43) Pub. Date: Nov. 22, 2012

(54) METAL OXIDE CONTAINING CATALYST FOR SIDE CHAIN ALKYLATION REACTIONS

(75) Inventor: Sivadinarayana Chinta, Missouri City, TX (US)

(73) Assignee: FINA TECHNOLOGY, INC., Houston, TX (US)

(21) Appl. No.: 13/457,506

(22) Filed: Apr. 27, 2012

Related U.S. Application Data
(60) Provisional application No. 61/488,778, filed on May 22, 2011.

(51) Int. Cl. C07C 2/88; B01J 29/08; B01J 21/12

(52) U.S. Cl. 585/437; 502/60; 502/73; 502/79; 502/74

(57) ABSTRACT

A catalyst containing a zeolite component and a metal oxide component, wherein the metal oxide component is ion-exchanged with the zeolite component resulting in an ion-modified zeolite, and wherein, under reaction conditions, the metal oxide component transforms into other oxide structures.

### CROSS-REFERENCE TO RELATED APPLICATIONS

This application claims priority to U.S. Provisional Patent No. 61/488,778 filed on May 22, 2011.

### FIELD

The present invention generally relates to metal-ion exchanged zeolites and zeolite-like materials. More specifically, the invention relates to zeolites having cesium oxide contained within the zeolite structure and used in the alkylation of toluene with methanol and/or formaldehyde to produce styrene and ethylbenzene.

### BACKGROUND

A zeolite is a crystalline alumino-silicate that is well known for its utility in several applications. It has been used in dealkylation, transalkylation, isomerization, cracking, disproportionation, and dewaxing processes, among others. Its well-ordered structure is composed of tetrahedral AlO₄⁻⁴ and SiO₄⁻⁴ molecules bound by oxygen atoms that form a system of pores typically on the order of 3 Å to 10 Å in diameter. These pores create a high internal surface area and allow the zeolite to selectively adsorb certain molecules while excluding others, based on the shape and size of the molecules. Thus, a zeolite can be categorized as a molecular sieve. A zeolite can also be referred to as a “shape selective catalyst.” The small pores can restrict reactions to certain transition states or certain products, preventing shapes that do not fit the contours or dimensions of the pores.

The pores in a zeolite are generally occupied by water molecules and cations. Cations balance out the negative charge caused by trivalent aluminum cations which are coordinated tetrahedrally by oxygen anions. A zeolite can exchange its native cations for other cations; one example is the exchange of sodium ions for ammonium ions. In some ion-exchanged forms, such as the hydrogen form of zeolite, the catalyst can be strongly acidic. For instance, zeolite can serve as a catalyst for Friedel-Crafts alkylations, replacing traditional aluminum trichloride and other liquid acid catalysts that can be corrosive and damaging to the reactor.

One alkylation reaction for which zeolite can be used as a catalyst is the alkylation of benzene with ethylene to form ethylbenzene. Ethylbenzene is an aromatic hydrocarbon with the chemical formula C₆H₅CH₂CH₃; it consists of a six-carbon aromatic ring with a single attached ethyl group. The ethylbenzene can then undergo a dehydrogenation reaction to form the monomer styrene, the monomer from which polystyrene is made. Polystyrene is a plastic that can form many useful products, including molded products and foamed products, all of which increase the need for production of styrene's precursor, ethylbenzene.

Other known processes to produce styrene include the alkylation of toluene. For instance, various alumina-silicate catalysts are utilized to react methanol and toluene to produce styrene. These processes allow for the production of styrene without the need for an intermediate step of obtaining ethylbenzene. However, such processes have been characterized by having very low yields in addition to having low selectivity to styrene. It would therefore be desirable to achieve a process for obtaining styrene without the need for an intermediate step of producing ethylbenzene. It would also be desirable to have a process for obtaining styrene that also has a high yield and selectivity to styrene.

### SUMMARY

An embodiment of the present invention is a catalyst including a zeolite component and an occluded metal oxide component. The occluded metal oxide component is contained within the framework of the zeolite component resulting in a modified zeolite that is capable of catalyzing the alkylation of toluene with a C1 source to produce styrene. Under reaction conditions the occluded metal oxide component is capable of increasing toluene conversion in an alkylation reaction of toluene with methanol.

In an embodiment, either alone or in combination with other embodiments, the occluded metal oxide component of the modified zeolite is capable of increasing selectivity to styrene in an alkylation reaction of toluene with a C1 source.

In an embodiment, either alone or in combination with other embodiments, the occluded metal oxide component of the modified zeolite increases the selectivity to styrene while decreasing the consumption of the C1 source.

In an embodiment, either alone or in combination with other embodiments, the occluded metal oxide component is selected from the group of cesium oxide, copper oxide, cerium oxide, and combinations thereof.

In an embodiment, either alone or in combination with other embodiments, the occluded metal oxide component makes up from 0.1% to 20% by weight of the modified zeolite.

In an embodiment, either alone or in combination with other embodiments, the occluded metal oxide component of the modified zeolite occluded metal oxide species is present in an amount of from 0.1 to 10 metal oxide species per unit cell of the zeolite.

In an embodiment, either alone or in combination with other embodiments, the zeolite is a faujasite type zeolite.

In an embodiment, either alone or in combination with other embodiments, the catalyst includes at least one promoter. The promoter can be selected from the group of Co, Mn, Ti, Zr, V, Nb, K, Cs, Ga, B, P, Rb, Ag, Na, Cu, Mg, Fe, Mo, Ce, and combinations thereof.

An embodiment of the present invention is a process for making styrene that includes reacting toluene with a C1 source in the presence of a zeolite catalyst in one or more reactors to form a product stream comprising styrene. The catalyst includes an occluded metal oxide component selected from the group of cesium oxide, copper oxide, cerium oxide, and combinations thereof, which improves toluene conversion.

In an embodiment, either alone or in combination with other embodiments, the C1 source is selected from the group of methanol, formaldehyde, formalin, trioxane, methylformcel, paraformaldehyde, methylal, dimethyl ether, and combinations thereof.

In an embodiment, either alone or in combination with other embodiments, the occluded metal oxide component of the modified zeolite occluded metal oxide species is present in an amount of from 0.1 to 10 metal oxide species per unit cell of the zeolite.

In an embodiment, either alone or in combination with other embodiments, the zeolite is a faujasite type zeolite.

In an embodiment, either alone or in combination with other embodiments, the catalyst includes at least one promoter selected from the group of Co, Mn, Ti, Zr, V, Nb, K, Cs, Ga, B, P, Rb, Ag, Na, Cu, Mg, Fe, Mo, Ce, and combinations thereof.

In an embodiment, either alone or in combination with other embodiments, the process has a toluene conversion of at least 5 mol %, optionally at least 10 mol %.

In an embodiment, either alone or in combination with other embodiments, the process has a styrene selectivity of at least 5 mol %, optionally at least 10 mol %.

In an embodiment, either alone or in combination with other embodiments, the process has a styrene selectivity plus ethylbenzene selectivity of at least 90 mol %.

The various embodiments of the present invention can be joined in combination with other embodiments of the invention and the listed embodiments herein are not meant to limit the invention. All combinations of embodiments of the invention are enabled, even if not given in a particular example herein.

### BRIEF DESCRIPTION OF DRAWINGS

FIG. 1 illustrates a flow chart for the production of styrene by the reaction of formaldehyde and toluene, wherein the formaldehyde is first produced in a separate reactor by either the dehydrogenation or oxidation of methanol and is then reacted with toluene to produce styrene.

FIG. 2 illustrates a flow chart for the production of styrene by the reaction of formaldehyde and toluene, wherein methanol and toluene are fed into a reactor, wherein the methanol is converted to formaldehyde and the formaldehyde is reacted with toluene to produce styrene.

FIG. 3 illustrates a fluidized bed reactor.

### DETAILED DESCRIPTION

The present invention relates to a metal ion modified species of a catalyst, such as a zeolite catalyst, to enhance conversion and product selectivity in an alkylation reaction. Specifically, a zeolite is modified by the addition of an occluded metal oxide, such as cesium oxide, copper oxide, or cerium oxide, in a way that results in improved conversion and product selectivity and inhibits unwanted by-product formation of an alkylation reaction.

As used herein, the term “metal ion” is meant to include all active metal ions and similar species, such as metal oxides, nanoparticles, and mixed metal oxide phases. Further, the term “ion-modified zeolite” as used herein refers to a zeolite that has been modified with a metal ion to enhance product selectivity. It is desirable that the metal ions not adversely affect the catalyst or cause significant by-product formation to occur.

The catalyst of the present invention may be supported by a zeolite or a zeolite like material. A zeolite is generally a porous, crystalline alumino-silicate, and it can be formed either naturally or synthetically. One method of forming synthetic zeolite is the hydrothermal digestion of silica, alumina, sodium or other alkyl metal oxide, and an organic templating agent. The amounts of each reactant and the inclusion of various metal oxides can lead to several different synthetic zeolite compositions. Furthermore, zeolite is commonly altered through a variety of methods to adjust characteristics such as pore size, structure, activity, acidity, and silica/alumina molar ratio. Thus, a number of different forms of zeolite are available.

Zeolite materials suitable for this invention may include silicate-based zeolites and amorphous compounds such as faujasites, mordenites, etc. Silicate-based zeolites are made of alternating SiO₄⁻ and MOₓ tetrahedra, where M is an element selected from the Groups 1 through 16 of the Periodic Table (new IUPAC). These types of zeolites have 4, 6, 8, 10, or 12-membered oxygen ring channels. An example of the zeolites of the present invention can include faujasites, such as an X-type or Y-type zeolite and zeolite beta. Zeolite-like materials can also be an effective substrate. Alternate molecular sieves also contemplated are zeolite-like materials such as the crystalline silicoaluminophosphates (SAPO) and the aluminophosphates (ALPO) and the like.

Another method of altering zeolite is by ion-exchange. Ion exchange may be performed by conventional ion exchange methods in which sodium, hydrogen, or other inorganic cations that may be typically present in a substrate are at least partially replaced via a fluid solution. In an embodiment, the fluid solution can include any medium that will solubilize the cation without adversely affecting the substrate. In an embodiment, the ion exchange is performed by heating a solution containing any promoter selected from the group of Co, Mn, Ti, Zr, V, Nb, K, Cs, Ga, B, P, Rb, Ag, Na, Cu, Mg, Fe, Mo, Ce, and any combinations thereof in which the promoter(s) is(are) solubilized in the solution, which may be heated, and contacting the solution with the substrate. In another embodiment, the ion exchange includes heating a solution containing any one selected from the group of Ce, Cu, P, Cs, B, Co, Ga, and any combinations thereof. In an embodiment, the solution is heated to temperatures ranging from 50 to 120° C. In another embodiment, the solution is heated to temperatures ranging from 80 to 100° C. Thus, a variety of zeolites and non-zeolites are available for use in conjunction with the present invention.

The various catalysts listed in the preceding paragraphs are not meant to be an exhaustive list, but is meant to indicate the type of catalysts that can be useful in the present invention. The choice of catalyst will depend on the reaction type and the reaction conditions in which it will be used. One skilled in the art can select any zeolite or non-zeolite catalyst that meets the needs of the intended reaction, provided that the catalyst increases the selectivity of the desired product and decreases unwanted side reactions.

The zeolites for use in this invention can include metal oxide species, such as for a non-limiting example cesium oxide species like Cs₂O. The metal oxide may be present within the structure of the zeolite, or support. The metal oxide present within the structure of the zeolite may be loosely contained within the structure of the zeolite. In an embodiment the metal oxide is not physically attached to the zeolite, but physically trapped within the zeolite cage structure, which can be referred to herein as occluded metal oxide or occluded cesium. In an embodiment occluded cesium oxide present in the structure of the zeolite can electrically influence the zeolite and alter its catalytic abilities.

In an embodiment occluded metal oxide species can be present in an amount of from 0.1 to 10 metal oxide species per unit cell of the zeolite or zeolite like material. Optionally the occluded metal oxide species can be present in an amount of from 1 to 7 metal oxide species per unit cell, optionally from 2 to 4 metal oxide species per unit cell.

In an embodiment occluded cesium oxide species can be present in an amount of from 0.1 to 10 Cs per unit cell of the zeolite or zeolite like material. Optionally the occluded cesium oxide can be present in an amount of from 1 to 7 Cs per unit cell, optionally from 2 to 4 Cs per unit cell.

In an embodiment occluded copper oxide species can be present in an amount of from 0.1 to 10 Cu per unit cell of the zeolite or zeolite like material. Optionally the occluded copper oxide can be present in an amount of from 1 to 7 Cu per unit cell, optionally from 2 to 4 Cu per unit cell.

In an embodiment occluded cerium oxide species can be present in an amount of from 0.1 to 10 Ce per unit cell of the zeolite or zeolite like material. Optionally the occluded cerium oxide can be present in an amount of from 1 to 7 Ce per unit cell, optionally from 2 to 4 Ce per unit cell.

In an embodiment the catalyst having occluded metal oxide in the support can further have additional metal ions added as a promoter on the support through a method such as ion exchange. The metal ions added through ion exchange are added by replacement of a cation of the support lattice, such as sodium or potassium, with the metal ion. In an embodiment the additional metal ions can range from 0.1 to 80% of the cations of the zeolite, optionally from 10 to 60% of the cations of the zeolite, optionally from 25 to 40% of the cations of the zeolite.

In an embodiment the catalyst having occluded cesium oxide in the support can further have additional cesium ions added as a promoter on the support through a method such as ion exchange. The cesium ions added through ion exchange are added by replacement of a cation of the support lattice, such as sodium or potassium. In an embodiment the additional cesium ions can range from 0.1 to 80% of the cations of the zeolite, optionally from 10 to 60% of the cations of the zeolite, optionally from 25 to 40% of the cations of the zeolite. In a like manner copper or cerium can be used as an occluded metal oxide and can have additional promoters added through ion exchange.

The catalyst of the present invention having occluded metal oxide in the support can increase the toluene conversion. However, the presence of the metal oxide may decrease the utilization of methanol. The methanol utilization may be increased by the addition of promoters. In an embodiment, the methanol utilization may be enhanced by the addition of boron (B) has a promoter. In an embodiment the boron in the catalyst can range from 0.01 wt % to 5 wt %, optionally from 0.1 wt % to 2 wt %, optionally from 0.4 wt % to 0.8 wt %.

In an embodiment the metal ion can be added to the zeolite in the amount of 0.1% to 50%, optionally 0.1% to 20%, optionally 0.1% to 5%, by weight of the zeolite. The metal ion can be added to the zeolite by any means known in the art. Generally, the method used is incipient wetness impregnation, wherein the metal ion precursor is added to an aqueous solution, which solution is poured over the zeolite. After sitting for a specified period, the zeolite is dried and calcined, such that the water is removed with the metal ion deposited on the zeolite surface. In an embodiment, the ion-modified zeolite can then be mixed with a binder by any means known in the art. The zeolite, or zeolite binder mixture, is shaped via extrusion or some other method into a form such as a pellet, tablet, cylinder, cloverleaf, dumbbell, symmetrical and asymmetrical polylobates, sphere, or any other shape suitable for the reaction bed. The shaped form is then usually dried and calcined. Drying can take place at a temperature of from 100° C. to 200° C. Calcining can take place at a temperature of from 400° C. to 900° C. in a substantially dry environment. The resultant catalyst aggregate can contain binder in concentrations of from 1% to 80%, optionally from 5% to 50%, optionally from 10% to 30%, by weight.

The powder form of zeolite and other catalysts may be unsuitable for use in the reactor, due to a lack of mechanical stability, making alkylation and other desired reactions difficult. To render a catalyst suitable for the reactor, it can be combined with a binder to form an aggregate, such as a zeolite aggregate, with enhanced mechanical stability and strength. The aggregate can then be shaped or extruded into a form suitable for the reaction bed. The binder can desirably withstand temperature and mechanical stress and ideally does not interfere with the reactants adsorbing to the catalyst. In fact, it is possible for the binder to form macropores, much greater in size than the pores of the catalyst, which provide improved diffusional access of the reactants to the catalyst.

Binder materials that are suitable for the present invention include, but are not limited to, silica, alumina, titania, zirconia, zinc oxide, magnesia, boria, silica-alumina, silica-magnesia, chromia-alumina, alumina-boria, silica-zirconia, silica gel, clays, similar species, and any combinations thereof. The most frequently used binders are amorphous silica and alumina, including gamma-, eta-, and theta-alumina. It should be noted that a binder can be used with many different catalysts, including various forms of zeolite and non-zeolite catalysts that require mechanical support.

The processes for which the ion-modified zeolite can be used include, but are not limited to, dehydrogenation, oxidation, reduction, adsorption, dimerization, oligomerization, polymerization, etherification, esterification, hydration, dehydration, condensation, acetalization, dealkylation, cyclization, alkylation, hydrodealkylation, transalkylation, isomerization, cracking, disproportionation, hydroisomerization, hydrocracking, aromatization, and any process employing a molecular sieve. One common process is alkylation and dehydrogenation.

Many different forms of alkylation reactions are possible. In general, alkylation occurs when an alkylating agent consisting of one or more carbon atoms is added to an alkylatable substrate. Alkylating agents that can be used in alkylation reactions are generally olefins. An olefin can be short chain, like ethylene, propylene, butene, and pentene, or it can be long chain with a higher number of carbon atoms. It can be an alpha olefin, an isomerized olefin, a branched-chain olefin or a mixture thereof. Alkylating agents other than olefins include alkynes, alkyl halides, alcohols, ethers, and esters. In some cases, the alkylating agent is diluted with a diluting agent prior to its introduction into the reaction bed. Especially for ethylene, diluting agents such as inert, or nonreactive, gases like nitrogen have been reported, with the concentration of the diluting agent greater than the concentration of the alkylating agent in the diluted feedstream, optionally around 70% diluting agent and 30% alkylating agent.

The alkylatable substrate is usually an unsaturated hydrocarbon or an aromatic. If the alkylatable substrate is an aromatic compound, it can be unsubstituted, monosubstituted, or polysubstituted, and it possesses at least one hydrogen atom bonded directly to the aromatic nucleus or some other site that will allow for alkylation to occur. The aromatic nucleus can be benzene or a compound having more than one aromatic ring, like naphthalene, anthracene, naphthacene, perylene, coronene, and phenanthrene. Compounds that have an aromatic character but contain a heteroatom in the ring can also be used, provided they will not cause unwanted side reactions. Substituents on the aromatic nucleus can be alkyl, hydroxy, alkoxy, aryl, alkaryl, aryloxy, cycloalkyl, halide, and/or other groups which do not interfere with the alkylation reaction and that have 1 to 20 carbon atoms. Aromatic substrates that may be alkylated by an alkylating agent include benzene, toluene, xylene, biphenyl, ethylbenzene, isopropylbenzene, normal propylbenzene, butylbenzene, pentylbenzene, hexylbenzene, heptylbenzene, octylbenzene, nonylbenzene, dodecylbenzene, pentadecylbenzene, hexyltoluene, nonyltoluene, dodecyltoluene, pentadecytoluene, alpha-methylnaphthalene, mesitylene, durene, cymene, pseudocumene, diethylbenzene, isoamylbenzene, isohexylbenzene, pentaethylbenzene, pentamethylbenzene, tetraethylbenzene, tetramethylbenzene, triethylbenzene, trimethylbenzene, butyltoluene, diethyltoluene, ethyltoluene, propyltoluene, dimethylnaphthalenes, ethylnaphthalene, dimethylanthracene, ethylanthracene, methylanthracene, dimethylphenanthrene, phenanthrenephenol, cresol, anisole, ethoxybenzene, propoxybenzene, butoxybenzene, pentoxybenzene, hexoxybenzene, any isomers thereof, and the like.

Another common alkylation reaction for which the present invention is useful is the alkylation of toluene with a C1 source, such as methanol. In an embodiments of the current invention, toluene is reacted with a C1 source to produce styrene and ethylbenzene. In an embodiment, the C1 source includes methanol or formaldehyde or a mixture of the two. In an alternative embodiment, toluene is reacted with one or more of the following: formalin (37-50 wt % H₂CO in solution of water and MeOH), trioxane (1,3,5-trioxane), methylformcel (55 wt % H₂CO in methanol), paraformaldehyde, methylal (dimethoxymethane), and dimethyl ether. In a further embodiment, the C1 source is selected from the group of methanol, formaldehyde, formalin, trioxane, methylformcel, paraformaldehyde, methylal, and dimethyl ether, and combinations thereof.

Formaldehyde can be produced either by the oxidation or dehydrogenation of methanol. Silver-based catalysts are most commonly used for the oxidation process but copper can also be used. Iron-molybdenum-oxide catalysts can be used for the dehydrogenation reaction. A separate process for the dehydrogenation or oxidation of methanol into formaldehyde gas can be utilized.

In an embodiment, formaldehyde is produced by the dehydrogenation of methanol to produce formaldehyde and hydrogen gas. This reaction step produces a dry formaldehyde stream that may be preferred, as it would not require the separation of the water prior to the reaction of the formaldehyde with toluene. Formaldehyde can also be produced by the oxidation of methanol to produce formaldehyde and water.

In the case of using a separate process to obtain formaldehyde, a separation unit may then be used in order to separate the formaldehyde from the hydrogen gas or water from the formaldehyde and unreacted methanol prior to reacting the formaldehyde with toluene for the production of styrene. This separation would inhibit the hydrogenation of the formaldehyde back to methanol. Purified formaldehyde could then be sent to styrene reactor and the unreacted methanol could be recycled.

Although the reaction has a 1:1 molar ratio of toluene and the C1 source, the ratio of the feedstreams is not limited within the present invention and can vary depending on operating conditions and the efficiency of the reaction system. If excess toluene or C1 source is fed to the reaction zone, the unreacted portion can be subsequently separated and recycled back into the process. In one embodiment the ratio of toluene:C1 source can range from between 100:1 to 1:100. In alternate embodiments the ratio of toluene:C1 source can range between from 50:1 to 1:50; from 20:1 to 1:20; from 10:1 to 1:10; from 5:1 to 1:5; from 2:1 to 1:2.

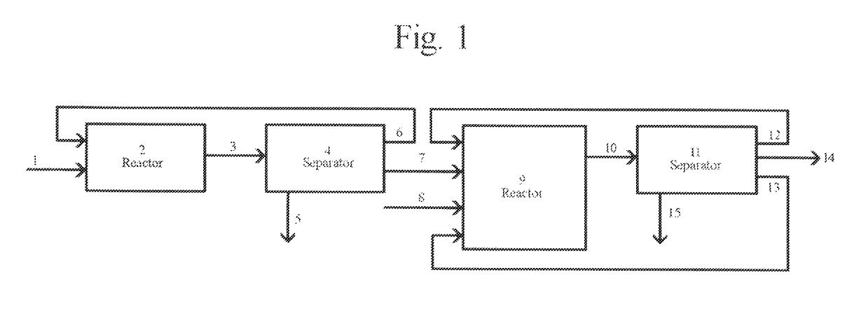

In FIG. 1 there is a simplified flow chart of one embodiment of the styrene production process described above. In this embodiment, a first reactor (2) is either a dehydrogenation reactor or an oxidation reactor. This reactor is designed to convert the first methanol feed (1) into formaldehyde. The gas product (3) of the reactor is then sent to a gas separation unit (4) where the formaldehyde is separated from any unreacted methanol and unwanted byproducts. Any unreacted methanol (6) can then be recycled back into the first reactor (2). The byproducts (5) are separated from the clean formaldehyde (7).

In one embodiment the first reactor (2) is a dehydrogenation reactor that produces formaldehyde and hydrogen and the separation unit (4) is a membrane capable of removing hydrogen from the product stream (3).

In an alternate embodiment the first reactor (2) is an oxidative reactor that produces product stream (3) including formaldehyde and water. The product stream (3) including formaldehyde and water can then be sent to the second reactor (9) without a separation unit (4).

The formaldehyde feed stream (7) is then reacted with a feed stream of toluene (8) in a second reactor (9). The toluene and formaldehyde react to produce styrene. The product (10) of the second reactor (9) may then be sent to an optional separation unit (11) where any unwanted byproducts (15) such as water can separated from the styrene, unreacted formaldehyde and unreacted toluene. Any unreacted formaldehyde (12) and the unreacted toluene (13) can be recycled back into the reactor (9). A styrene product stream (14) can be removed from the separation unit (11) and subjected to further treatment or processing if desired.

The operating conditions of the reactors and separators can be system specific and can vary depending on the feedstream composition and the composition of the product streams. The reactor (9) for the reactions of methanol to formaldehyde and toluene with formaldehyde will operate at elevated temperatures and pressures and may contain a basic or neutral catalyst system. The temperature can range in a non-limiting example from 250° C. to 750° C., optionally from 350° C. to 550° C., optionally from 375° C. to 475° C. The pressure can range in a non-limiting example from 0.1 atm to 70 atm, optionally from 0.1 atm to 10 atm, optionally from 0.1 atm to 3 atm.

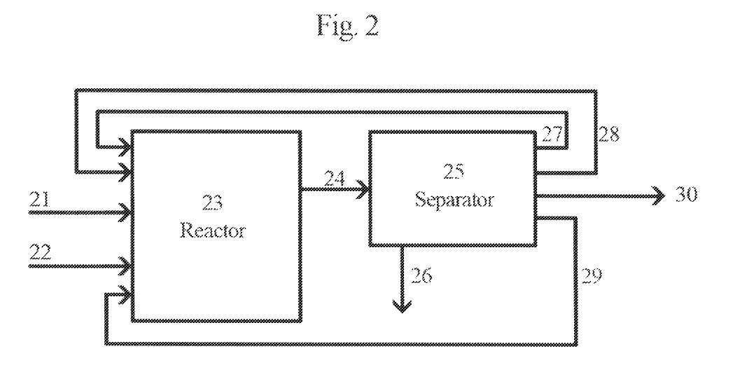

FIG. 2 is a simplified flow chart of another embodiment of the styrene process discussed above. A methanol containing feed stream (21) is fed along with a feed stream of toluene (22) in a reactor (23). The methanol reacts with a catalyst in the reactor to produce formaldehyde. The toluene and formaldehyde then react to produce styrene. The product (24) of the reactor (23) may then be sent to an optional separation unit (25) where any unwanted byproducts (26) can separated from the styrene, unreacted methanol, unreacted formaldehyde and unreacted toluene. Any unreacted methanol (27), unreacted formaldehyde (28) and the unreacted toluene (29) can be recycled back into the reactor (23). A styrene product stream (30) can be removed from the separation unit (25) and subjected to further treatment or processing if desired.

The operating conditions of the reactors and separators will be system specific and can vary depending on the feedstream composition and the composition of the product streams. The reactor (23) for the reactions of methanol to formaldehyde and toluene with formaldehyde will operate at elevated temperatures and pressures and may contain a basic or neutral catalyst system. The temperature can range in a non-limiting example from 250° C. to 750° C., optionally from 350° C. to 550° C., optionally from 375° C. to 475° C. The pressure can range in a non-limiting example from 0.1 atm to 70 atm, optionally from 0.1 atm to 10 atm, optionally from 0.1 atm to 3 atm.

Inert diluents such as helium and nitrogen may be included in the feed to adjust the gas partial pressures. Optionally, CO₂ or water (steam) can be included in the feed stream as these components may have beneficial properties, such as in the prevention of coke deposits. The reaction pressure is not a limiting factor regarding the present invention and any suitable condition is considered to be within the scope of the invention.

In the coupling reaction of toluene and formaldehyde in the present invention, short reaction times have improved the conversion of toluene. These short reaction times improve the conversion of toluene relative to that when the catalyst has been on stream for long periods of time. In an embodiment, the contact times of the reactants with the catalyst range from about 0.01 to about 5 seconds. In a further embodiment, the contact times range from about 0.1 to about 3 seconds.

Any suitable space velocity can be considered to be within the scope of the invention. The space velocity ranges given are not limiting on the present invention and any suitable condition is considered to be within the scope of the invention.

In addition, modification of the physical character of the catalyst to enhance the diffusion rate of the reactants to active sites and the products away from active sites would be advantageous to the conversion of reactants and selectivity of desired products. Such catalyst modifications include depositing the active components onto an inert substrate, optimizing the size of catalyst particles, and imparting void areas throughout the catalyst. Increasing porosity and/or increasing the surface area of the catalyst can accomplish this optimization.

Embodiments of reactors that can be used with the present invention can include, by non-limiting examples: fixed bed reactors; fluid bed reactors; moving bed reactors; and entrained bed reactors. Reactors capable of the elevated temperature and pressure as described herein, and capable of enabling contact of the reactants with the catalyst, can be considered within the scope of the present invention. Embodiments of the particular reactor system may be determined based on the particular design conditions and throughput, as by one of ordinary skill in the art, and are not meant to be limiting on the scope of the present invention.

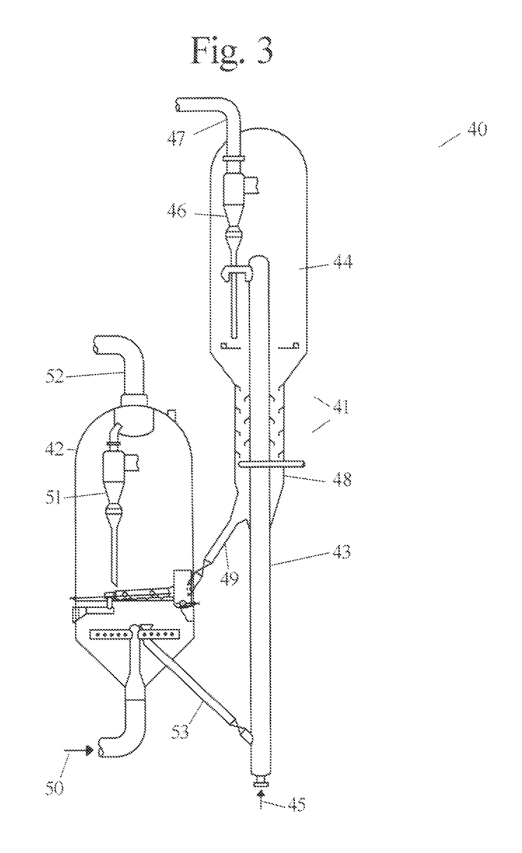

An example of a fluidized bed reactor having catalyst regeneration capabilities that may be employed with the present invention is illustrated in FIG. 3. This type of reactor system employing a riser can be modified as needed, for example by insulating or heating the riser if thermal input is needed, or by jacketing the riser with cooling water if thermal dissipation is required. These designs can also be used to replace catalyst while the process is in operation, by withdrawing catalyst from the regeneration vessel from an exit line (not shown) or adding new catalyst into the system while in operation. The riser reactor can be replaced with a downer reactor (not shown). In an embodiment (not shown), the reaction zone includes both riser and downer reactors.

FIG. 3 is a schematic illustration of an embodiment of the present invention having the capability for continuous reaction with catalyst regeneration. The reactor system (40) generally includes two main zones for reaction (41) and regeneration (42). A reaction zone can have a vertical conduit, or riser (43), as the main reaction site, with the effluent of the conduit emptying into a large volume process vessel, which may be referred to as a separation vessel (44). In the reaction riser (43), a feed stream (45), such as toluene and methanol, is contacted with a fluidized catalyst, which can be a relatively large fluidized bed of catalyst, at reactor conditions. The residence time of catalyst and hydrocarbons in the riser (43) needed for substantial completion of the reaction may vary as needed for the specific reactor design and throughput design. The flowing vapor/catalyst stream leaving the riser (43) may pass from the riser to a solids-vapor separation device, such as a cyclone (46), normally located within and at the top of the separation vessel (44). The products of the reaction can be separated from the portion of catalyst that is carried by the vapor stream by means of one or more cyclone (46) and the products can exit the cyclone (46) and separation vessel (44) via line (47). The spent catalyst falls downward to a stripper (48) located in a lower part of the separation vessel (44). Catalyst can be transferred to a regeneration vessel (42) by way of a conduit (49) connected to the stripper (48).

The catalyst can be continuously circulated from the reaction zone (41) to the regeneration vessel (42) and then again to the reaction zone (41). The catalyst can therefore act as a vehicle for the transfer of heat from zone to zone as well as providing the necessary catalytic activity. Catalyst from the reaction zone (41) that is being transferred to the regeneration zone (42) can be referred to as “spent catalyst”. The term “spent catalyst” is not intended to be indicative of a total lack of catalytic activity by the catalyst particles. Catalyst, which is being withdrawn from the regeneration vessel (42), is referred to as “regenerated” catalyst. The catalyst can be regenerated in the regeneration vessel (42) by heat and contact with a regeneration stream (50). The regeneration stream (50) can include oxygen and can include steam. The regenerated catalyst can be separated from the regeneration stream by the use of one or more cyclones (51) that can enable the removal of the regeneration vessel (42) via line (52). The regenerated catalyst can be transferred via line (53) to the lower section of the riser (43) where it is again in contact with the feed stream (45) and can flow up the riser (43).

In an embodiment, the reactants may be injected into the reactor(s) in a stage-wise manner. The fluidized bed reaction zone may contain a top section, a bottom section, and an intermediate section, having a span that reaches between the top section and the bottom section. The toluene feed may be injected at any point, or points, along the fluidized bed. The C1 source, which may include formaldehyde, may also be injected at any point, or points, along the fluidized bed. In an embodiment, the toluene feed is injected downstream from the C1 source injection point. In another embodiment, the C1 source is injected downstream from the toluene feed injection point. In a further embodiment, both the C1 source and the toluene feed are injected at the same point along the fluidized bed. In an embodiment, the fluidized bed is a dense bed fluidized reactor.

In another embodiment, the one or more reactors may include one or more catalyst beds. In the event of multiple beds, an inert material layer can separate each bed. The inert material can include any type of inert substance. In an embodiment, a reactor includes between 1 and 10 catalyst beds. In a further embodiment, a reactor includes between 2 and 5 catalyst beds. In addition, the C1 source and toluene may be injected into a catalyst bed, an inert material layer, or both. In a further embodiment, at least a portion of the C1 source is injected into a catalyst bed(s) and at least a portion of the toluene feed is injected into an inert material layer(s). In an even further embodiment, the entire C1 source is injected into at a catalyst bed(s) and all of the toluene feed is injected into an inert material layer(s). In another embodiment, at least a portion of the toluene feed is injected into a catalyst bed(s) and at least a portion the C1 source is injected into an inert material layer(s). In a further embodiment, the toluene feed is injected prior to the first catalyst bed while at least a portion of the C₁ source and/or at least a portion of the co-feed are injected into one or more catalyst bed(s) along the reactor to control the toluene: C₁ source in each catalyst bed.

The toluene and C1 source coupling reaction may have a toluene conversion percent greater than 0.01 wt %. In an embodiment the toluene and C1 source coupling reaction is capable of having a toluene conversion percent in the range of from about 0.05 wt % to about 50 wt %. In a further embodiment the toluene and C1 source coupling reaction is capable of having a toluene conversion in the range of from about 2 wt % to about 20 wt %.

In an embodiment the toluene and C1 source coupling reaction is capable of selectivity to styrene up to about 85 wt %. In another embodiment, the toluene and formaldehyde coupling reaction is capable of selectivity to styrene in the range of from about 60 wt % to about 80 wt %. In an embodiment the toluene to formaldehyde coupling reaction is capable of selectivity to ethylbenzene in the range of from about 10 wt % to about 50 wt %. In another embodiment, the toluene to formaldehyde coupling reaction is capable of selectivity to ethylbenzene in the range of from about 15 wt % to about 35 wt %. In an embodiment, the ratio of selectivity to styrene and selectivity to ethylbenzene (S_{sty}:S_{EB}) is in the range of from about 1:5 to about 3:5.

### EXAMPLES

### Example 1

A zeolite based catalyst was promoted with Cs (both ion-exchange and occluded) to make four Cs promoted catalysts, labeled A, B, C, D, having varying Cs content. The Cs content was as follows: A>B>C>D. The catalysts were tested in a lab scale reactor on the ability to catalyze the alkylation of toluene with methanol. The results are listed in Table 1 and indicate that the toluene conversion increased as the Cs content increased, but the selectivity to styrene decreased as the Cs content increased.

Procedure used to produce the cesium ion-exchanged zeolite material: A glass cylinder (2″ inside diameter), fitted with a sintered glass disk and stopcock at the lower end, was charged with 544-HP zeolite (100 g, W.R. Grace) and CsOH (400 mL, 1.0 M in water). The mixture was then brought to 90° C. and allowed to stand for 4 h. The liquid was drained from the zeolite material and another aliquot of CsOH (400 mL of 1.0 M solution in water) was added and allowed to stand for 3 hours at 90° C. The liquid was drained from the zeolite material and another aliquot of CsOH (400 mL of 1.0 M solution in water) was added and allowed to stand for 15 hours at 90° C. The liquid was drained from the zeolite material and dried at 150° C. for 1.5 hours.

This procedure was repeated to produce a cesium ion-exchanged zeolite material having a Cs content as follows: A>B>C>D.

Conversion of toluene increased with additional Cs content, along with the selectivity to ethylbenzene. Selectivity to cumene and alpha methyl styrene remained within acceptable ranges.

TABLE 1

| | Time On | | | | | | | | |
|---|---|---|---|---|---|---|---|---|---|
| | Stream | | | | | | | | |
| Catalyst | (hh:mm) | X_{Tol} | S_{Bz} | S_{Xyl} | S_{EB} | S_{Sty} | S_{Cumene} | Sₐₘₛ | X_{MeOH} |
| A | 3:19 | 13.2 | 0.2 | 0.2 | 91.1 | 3.9 | 3.84 | 0.2 | 45.1 |
| | 4:30 | 12.4 | 0.2 | 0.2 | 91.1 | 5.0 | 3.07 | 0.2 | 43.9 |
| | 5:29 | 11.3 | 0.2 | 0.2 | 91.4 | 4.9 | 2.85 | 0.2 | 42.5 |
| | 7:01 | 10.0 | 0.2 | 0.3 | 93.1 | 4.1 | 2.21 | 0.1 | 40.0 |
| B | 2:50 | 12.3 | 0.2 | 0.3 | 87.7 | 5.3 | 5.0 | 0.5 | 52.6 |
| | 3:44 | 11.4 | 0.2 | 0.3 | 88.0 | 5.9 | 4.4 | 0.5 | 51.3 |
| | 4:21 | 9.9 | 0.3 | 0.3 | 89.0 | 5.2 | 4.2 | 0.4 | 50.2 |
| C | 3:29 | 11.8 | 0.2 | 0.2 | 88.2 | 8.0 | 2.87 | 0.3 | 34.0 |
| | 5:46 | 10.6 | 0.2 | 0.2 | 89.4 | 7.2 | 2.42 | 0.3 | 31.5 |
| | 6:26 | 9.6 | 0.3 | 0.2 | 91.2 | 6.0 | 2.09 | 0.2 | 29.8 |
| D | 3:20 | 7.8 | 0.3 | 0.4 | 71.8 | 24.7 | 2.2 | 0.6 | 11.4 |
| | 4:58 | 6.1 | 0.4 | 0.4 | 76.4 | 20.5 | 1.9 | 0.3 | 14.2 |
| | 5:58 | 5.8 | 0.4 | 0.4 | 76.7 | 20.3 | 1.8 | 0.3 | 13.5 |

### Example 2

To examine the effect of the addition of boron on a catalyst having cesium promoters (both ion-exchange and occluded) such as in Example 1 above, samples of catalyst A were then treated to add boron to produce catalyst E having 0.3 wt % boron content; catalyst F having 0.6 wt % boron content; and catalyst F having 0.9 wt % boron content. Catalyst E, F, and G were tested in a lab scale reactor on the ability to catalyze the alkylation of toluene with methanol. The results are listed in Table 2 and indicate that the toluene conversion increased as the boron content increased. Also as the boron content increased the methanol conversion decreased, indicating more efficient methanol utilization. Catalyst F having a 0.6 wt % boron content resulted in the highest selectivity to styrene and the highest conversion of toluene, indicating a synergistic effect of Cs and a boron content of 0.6 wt %.

Deposition of 0.3 wt % boron onto cesium ion-exchanged zeolite material: The cesium ion-exchanged zeolite material (35 g) was treated with a solution of boric acid (0.6 g) dissolved in acetone (500 mL) at room temperature for 2 hours. The (Cs, B)/X material was then dried at 110° C. for 20 hours.

Deposition of 0.6 wt % boron onto cesium ion-exchanged zeolite material: The cesium ion-exchanged zeolite material (35 g) was treated with a solution of boric acid (1.2 g) dissolved in acetone (500 mL) at room temperature for 2 hours. The (Cs, B)/X material was then dried at 110° C. for 20 hours.

Deposition of 0.9 wt % boron onto cesium ion-exchanged zeolite material: The cesium ion-exchanged zeolite material (35 g) was treated with a solution of boric acid (1.8 g) dissolved in acetone (500 mL) at room temperature for 2 hours. The (Cs, B)/X material was then dried at 110° C. for 20 hours.

TABLE 2

| | Time On | | | | | | | | |
|---|---|---|---|---|---|---|---|---|---|
| | Stream | | | | | | | | |
| Catalyst | (hh:mm) | X_{Tol} | S_{Bz} | S_{Xyl} | S_{EB} | S_{Sty} | S_{Cumene} | Sₐₘₛ | X_{MeOH} |
| A | 3:19 | 13.2 | 0.2 | 0.2 | 91.1 | 3.9 | 3.8 | 0.2 | 45.1 |
| | 4:30 | 12.4 | 0.2 | 0.2 | 91.1 | 5.0 | 3.1 | 0.2 | 43.9 |
| | 5:29 | 11.3 | 0.2 | 0.2 | 91.4 | 4.9 | 2.9 | 0.2 | 42.5 |
| | 7:01 | 10.0 | 0.2 | 0.3 | 93.1 | 4.1 | 2.2 | 0.1 | 40.0 |
| E | 3:19 | 15.1 | 0.2 | 0.2 | 86.5 | 8.2 | 4.4 | 0.4 | 30.7 |
| | 4:12 | 15.1 | 0.2 | 0.2 | 86.9 | 7.2 | 4.9 | 0.4 | 29.1 |
| | 4:37 | 15.1 | 0.2 | 0.2 | 87.8 | 6.5 | 4.6 | 0.4 | 29.5 |
| | 4:58 | 14.7 | 0.2 | 0.2 | 87.8 | 6.6 | 4.5 | 0.4 | 29.1 |
| | 6:03 | 13.6 | 0.2 | 0.2 | 89.1 | 5.6 | 4.2 | 0.3 | 27.0 |
| F | 2:54 | 16.1 | 0.2 | 0.2 | 74.0 | 22.0 | 2.7 | 0.6 | 33.1 |
| | 3:31 | 17.7 | 0.2 | 0.2 | 79.9 | 14.4 | 4.2 | 0.8 | 28.8 |
| | 4:18 | 17.4 | 0.2 | 0.2 | 80.9 | 13.0 | 4.5 | 0.9 | 30.2 |
| | 5:22 | 12.4 | 0.2 | 0.2 | 88.1 | 7.0 | 3.8 | 0.3 | 25.0 |
| G | 4:15 | 16.9 | 0.2 | 0.1 | 89.2 | 4.1 | 5.8 | 0.2 | |
| | 5:11 | 17.7 | 0.2 | 0.1 | 89.5 | 3.8 | 5.8 | 0.2 | |
| | 5:34 | 16.5 | 0.2 | 0.1 | 89.8 | 3.7 | 5.6 | 0.2 | |
| | 5:57 | 16.1 | 0.2 | 0.1 | 90.8 | 3.5 | 5.0 | 0.2 | |

The term “conversion” refers to the percentage of reactant (e.g. toluene) that undergoes a chemical reaction.

X_{Tol}=conversion of toluene (mol %)=(Tolᵢₙ−Tolₒᵤₜ)/Tolᵢₙ

X_{MeOH}=conversion of methanol to styrene+ethylbenzene (mol %)

The term “selectivity” refers to the relative activity of a catalyst in reference to a particular compound in a mixture. Selectivity is quantified as the proportion of a particular product relative to all other products.

S_{Sty}=selectivity of toluene to styrene (mol %)=Styₒᵤₜ/Tol_{converted}

S_{Bz}=selectivity of toluene to benzene (mol %)=Benzeneₒᵤₜ/Tol_{converted}

S_{EB}=selectivity of toluene to ethylbenzene (mol %)=EBₒᵤₜ/Tol_{converted}

S_{Xyl}=selectivity of toluene to xylenes (mol %)=Xylenesₒᵤₜ/Tol_{converted}

S_{sty+EB}(MeOH)=selectivity of methanol to styrene+ethylbenzene (mol %)=(Styₒᵤₜ+EBₒᵤₜ)/MeOH_{converted}

The term “deactivated catalyst” refers to a catalyst that has lost enough catalyst activity to no longer be efficient in a specified process. Such efficiency is determined by individual process parameters.

The term “ion-modified binder” as used herein refers to a binder for a catalyst that has been modified with a metal ion.

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

The term “regenerated catalyst” refers to a catalyst that has regained enough activity to be efficient in a specified process. Such efficiency is determined by individual process parameters.

The term “regeneration” refers to a process for renewing catalyst activity and/or making a catalyst reusable after its activity has reached an unacceptable/inefficient level. Examples of such regeneration may include passing steam over a catalyst bed or burning off carbon residue, for example.

The term “zeolite” refers to a molecular sieve containing a silicate lattice, usually in association with some aluminum, boron, gallium, iron, and/or titanium, for example. In the following discussion and throughout this disclosure, the terms molecular sieve and zeolite will be used more or less interchangeably. One skilled in the art will recognize that the teachings relating to zeolites are also applicable to the more general class of materials called molecular sieves.

The various embodiments of the present invention can be joined in combination with other embodiments of the invention and the listed embodiments herein are not meant to limit the invention. All combinations of various embodiments of the invention are enabled, even if not given in a particular example herein.

While illustrative embodiments have been depicted and described, modifications thereof can be made by one skilled in the art without departing from the spirit and scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the embodiments disclosed herein are usable and combinable with every other embodiment disclosed herein, and consequently, this disclosure is enabling for any and all combinations of the embodiments disclosed herein. Other and further embodiments, versions and examples of the invention may be devised without departing from the basic scope thereof and the scope thereof is determined by the claims that follow.

What is claimed is:

1. A catalyst comprising:
a zeolite component; and
an occluded metal oxide component;
wherein the occluded metal oxide component is contained within the framework of the zeolite component resulting in a modified zeolite;
wherein the catalyst is capable of catalyzing the alkylation of toluene with a C1 source to produce styrene;
wherein under reaction conditions, the occluded metal oxide component is capable of increasing toluene conversion in an alkylation reaction of toluene with a C1 source.

2. The catalyst of claim 1, wherein the occluded metal oxide component of the modified zeolite is capable of increasing selectivity to styrene in an alkylation reaction of toluene with a C1 source.

3. The catalyst of claim 1, wherein the occluded metal oxide component of the modified zeolite increases the selectivity to styrene while decreasing the consumption of the C1 source.

4. The catalyst of claim 1, wherein the occluded metal oxide component is selected from the group consisting of cesium oxide, copper oxide, cerium oxide, and combinations thereof.

5. The catalyst of claim 1, wherein the occluded metal oxide component makes up from 0.1% to 20% by weight of the modified zeolite.

6. The catalyst of claim 1, wherein the occluded metal oxide component of the modified zeolite occluded metal oxide species is present in an amount of from 0.1 to 10 metal oxide species per unit cell of the zeolite.

7. The catalyst of claim 1, wherein the zeolite is a faujasite type zeolite.

8. The catalyst of claim 1, further comprising at least one promoter.

9. The catalyst of claim 8, wherein the at least one promoter is selected from the group consisting of Co, Mn, Ti, Zr, V, Nb, K, Cs, Ga, B, P, Rb, Ag, Na, Cu, Mg, Fe, Mo, Ce, and combinations thereof.

10. A process for making styrene comprising:
reacting toluene with a C1 source in the presence of a zeolite catalyst in one or more reactors to form a product stream comprising styrene;
wherein the zeolite catalyst comprises an occluded metal oxide component, which improves toluene conversion;
wherein the occluded metal oxide component is selected from the group consisting of cesium oxide, copper oxide, cerium oxide, and combinations thereof.

11. The process of claim 10, wherein the C1 source is selected from the group consisting of methanol, formaldehyde, formalin, trioxane, methylformcel, paraformaldehyde, methylal, dimethyl ether, and combinations thereof.

12. The process of claim 10, wherein the occluded metal oxide component of the modified zeolite occluded metal oxide species is present in an amount of from 0.1 to 10 metal oxide species per unit cell of the zeolite.

13. The process of claim 10, wherein the zeolite is a faujasite type zeolite.

14. The process of claim 10, further comprising at least one promoter.

15. The process of claim 10, wherein the at least one promoter is selected from the group consisting of Co, Mn, Ti, Zr, V, Nb, K, Cs, Ga, B, P, Rb, Ag, Na, Cu, Mg, Fe, Mo, Ce, and combinations thereof.

16. The process of claim 10, having a toluene conversion of at least 5 mol %.

17. The process of claim 10, having a toluene conversion of at least 10 mol %.

18. The process of claim 10, having a styrene selectivity of at least 5 mol %.

19. The process of claim 10, having a styrene selectivity of at least 10 mol %.

20. The process of claim 10, having a styrene selectivity plus ethylbenzene selectivity of at least 90 mol %.
